(19) United States
(12) Patent Application Publication
(10) Pub. No.: US 2025/0161021 A1
(43) Pub. Date: May 22, 2025

(54) TISSUE ANCHOR

(71) Applicants: Smith & Nephew, Inc., Memphis, TN (US); Smith & Nephew Orthopaedics AG, Zug (CH); Smith & Nephew Asia Pacific Pte. Limited

(72) Inventors: Luis Alejandro Miranda Cruz, San Francisco (CR); Elias Milad Sibani, Alajuela (CR)

(73) Assignees: Smith & Nephew, Inc., Memphis, TN (US); Smith & Nephew Orthopaedics AG, Zug (CH); Smith & Nephew Asia Pacific Pte. Limited, (omitted)

(21) Appl. No.: 19/035,460

(22) Filed: Jan. 23, 2025

Related U.S. Application Data
(63) Continuation of application No. PCT/US2023/028455, filed on Jul. 24, 2023.
(60) Provisional application No. 63/392,355, filed on Jul. 26, 2022.

(51) Int. Cl. A61F 2/08

(52) U.S. Cl. CPC A61F 2/0811; A61F 2002/0817; A61F 2002/0847; A61F 2220/0016

(57) ABSTRACT

A tissue anchor delivery system includes an elongate shaft and a tissue anchor magazine coupled to the elongate shaft. The delivery system includes a plurality of tissue anchors arranged sequentially within the tissue anchor magazine in a first, pre-deployed configuration. An actuation assembly is configured to deploy a leading one of the tissue anchors in a distal direction from the distal end of the elongate shaft, wherein upon deployment, the deployed tissue anchor transitions from the first, pre-deployed configuration to a second, deployed configuration. Each of the tissue anchors including a tab positioned near the proximal end of the anchor body and a barb positioned near the distal end of the anchor body. The tab and the barb may move relative to the anchor body between the pre-deployed configuration and the deployed configuration.

### CROSS REFERENCE TO RELATED APPLICATIONS

This application is a continuation of International Application No. PCT/US2023/028455, filed Jul. 24, 2023, which claims the benefit of and priority to U.S. Provisional Patent Application Ser. No. 63/392,355 filed on Jul. 26, 2022, the contents of which are incorporated herein by reference.

### TECHNICAL FIELD

The present disclosure pertains generally, but not by way of limitation, to orthopedic implants and methods of treatment. More particularly, the present disclosure relates to methods and apparatus for delivery and fixation of sheet-like materials, such as for treating a full or partial thickness tear of a tendon, such as the supraspinatus tendon of the shoulder or other anatomical joint.

### BACKGROUND

With its complexity, range of motion and extensive use, a common soft tissue injury is damage to the rotator cuff or rotator cuff tendons. Damage to the rotator cuff is a potentially serious medical condition that may occur during hyperextension, from an acute traumatic tear or from overuse of the joint. Current procedures for treatment of a torn tendon include affixing a biocompatible implant over the torn tendon. There is an ongoing need to deliver and adequately secure medical implants during an arthroscopic procedure in order to treat injuries to the rotator cuff, rotator cuff tendons, or other soft tissue or tendon injuries throughout a body.

### BRIEF SUMMARY

This disclosure provides design, material, manufacturing method, and use alternatives for medical devices. An example tissue anchor may include an anchor body having a proximal end and a distal end, wherein the distal end includes a distal tip, a tab positioned near the proximal end of the anchor body, wherein the tab is movable from a first position to a second position, and a first barb positioned near the distal end of the anchor body, wherein the first barb is movable from a first position to a second position. The first barb is biased toward the second position, such that when an applied force is removed from the first barb, the first barb automatically reverts to the second position.

Alternatively or additionally to any of the embodiments above, the first barb may be in alignment with the anchor body when in the first position.

Alternatively or additionally to any of the embodiments above, the first barb may deflect away from the anchor body when in the second position.

Alternatively or additionally to any of the embodiments above, the tab may be in alignment with the anchor body when in the first position.

Alternatively or additionally to any of the embodiments above, the tab may deflect away from the anchor body when in the second position.

Alternatively or additionally to any of the embodiments above, the tab may be biased toward the second position, such that when an applied force is removed from the tab, the tab automatically reverts to the second position.

Alternatively or additionally to any of the embodiments above, when the tab is in the second position and the first barb is in the second position, the tab may deflect away from the anchor body in a first direction and the first barb may deflect away from the anchor body in a second direction different from the first direction.

Alternatively or additionally to any of the embodiments above, a second barb may be positioned near the distal end of the anchor body, wherein the second barb includes a first position in which the second barb is in alignment with the anchor body, and a second position in which the second barb deflects away from the anchor body.

Alternatively or additionally to any of the embodiments above, the second barb may be biased toward the second position, such that when an applied force is removed from the second barb, the second barb automatically reverts to the second position.

Alternatively or additionally to any of the embodiments above, the first and second barbs may be located on opposite sides of the anchor body.

Alternatively or additionally to any of the embodiments above, the tissue anchor may be a monolithic structure and the tab includes a first base end and a second free end, the first base end being joined to the anchor body, and the first barb includes a first base end and a second free end, the first base end being joined to the anchor body.

Alternatively or additionally to any of the embodiments above, the first barb may be cut out from the anchor body such that the anchor body includes an opening, wherein the first barb is located within a perimeter of the opening in the first position and the first barb extends outward from an opening in the second position.

An example tissue anchor delivery system may include an elongate shaft having a longitudinal axis, the elongate shaft having a lumen extending therein to a distal end of the elongate shaft, and a tissue anchor magazine coupled to the elongate shaft. The delivery system may include a plurality of tissue anchors each including a front side and a back side, the plurality of tissue anchors arranged sequentially within the tissue anchor magazine in a first, pre-deployed configuration, and an actuation assembly configured to deploy a leading one of the plurality of tissue anchors in a distal direction from the distal end of the elongate shaft, wherein upon deployment, the leading one of the plurality of tissue anchors transitions from the first, pre-deployed configuration to a second, deployed configuration.

Alternatively or additionally to any of the embodiments above, the plurality of tissue anchors may be arranged front-to-back within the tissue anchor magazine such that the back side of the leading one of the plurality of tissue anchors is facing the front side of an adjacent one of the plurality of tissue anchors.

Alternatively or additionally to any of the embodiments above, the front side of the adjacent one of the plurality of tissue anchors may face the longitudinal axis and moves toward the longitudinal axis after deployment of the leading one of the plurality of tissue anchors.

Alternatively or additionally to any of the embodiments above, the actuation assembly may include a pusher positioned within the lumen of the elongate shaft and a trigger, wherein actuation of the trigger actuates the pusher to move along a longitudinal axis to engage the proximal end of the leading one of the plurality of tissue anchors to deploy the leading one of the plurality of tissue anchors.

Alternatively or additionally to any of the embodiments above, upon deployment of the leading one of the plurality of tissue anchors, the plurality of tissue anchors remaining in the tissue anchor magazine may shift toward the longitudinal axis of the pusher such that another one of the plurality of tissue anchors moves within the lumen of the elongate shaft.

Alternatively or additionally to any of the embodiments above, each of the plurality of tissue anchors may include an anchor body having a proximal end and a distal end, wherein the distal end includes a distal tip, a tab positioned near the proximal end of the anchor body, and a first barb positioned near the distal end of the anchor body, wherein the tab and the first barb move relative to the anchor body when transitioning from the first, pre-deployed configuration to the second, deployed configuration.

Another example tissue anchor may include an anchor body, a first arm having a proximal end and a distal end, a second arm having a proximal end and a distal end, and a third arm having a proximal end and a distal end, wherein the proximal ends of the first, second, and third arms are coupled to the anchor body and the first, second, and third arms extend distally from the anchor body. The tissue anchor may further include a first prong connected to the distal end of the first arm, the first prong having an aperture extending therein, a second prong connected to the distal end of the second arm, the second prong having an aperture extending therein, and a third prong connected to the distal end of the third arm, the third prong having an aperture extending therein, wherein the first arm, the second arm, and the third arm are symmetrically arranged about the anchor body.

Alternatively or additionally to any of the embodiments above, a distal end of the first prong may be pointed, a distal end of the second prong may be pointed, and a distal end of the third prong may be pointed.

Alternatively or additionally to any of the embodiments above, the aperture of the first prong, the aperture of the second prong, and the aperture of the third prong may each be configured to receive a tine of a tissue anchor delivery device therein.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 illustrates an anterior view of a patient including a cross-section of a shoulder;

FIG. 2 illustrates the shoulder including a humerus and a scapula of FIG. 1;

FIG. 3 illustrates a tendon with a tendon repair implant fixed thereto with an exemplary tissue anchor;

FIG. 4 illustrates a perspective view of an exemplary tissue anchor in a pre-deployed configuration;

FIG. 5 illustrates a side view of the exemplary tissue anchor of FIG. 4 in the pre-deployed configuration;

FIG. 6A illustrates a perspective view of the exemplary tissue anchor of FIG. 4 in a deployed configuration;

FIG. 6B illustrates a front view of the exemplary tissue anchor of FIG. 6A in the deployed configuration;

FIG. 6C illustrates a side view of the exemplary tissue anchor of FIG. 6A in the deployed configuration;

FIG. 7A illustrates a perspective view of an exemplary tissue anchor in a deployed configuration;

FIG. 7B illustrates a front view of the exemplary tissue anchor of FIG. 7A in the deployed configuration;

FIG. 7C illustrates a side view of the exemplary tissue anchor of FIG. 7A in the deployed position;

FIG. 8A illustrates a perspective view of an exemplary tissue anchor in a pre-deployed configuration;

FIG. 8B illustrates a perspective view of the exemplary tissue anchor of FIG. 8A in a deployed configuration, wherein a first barb and a second barb deflect away from the tissue anchor in opposite directions;

FIG. 8C illustrates a perspective view of the exemplary tissue anchor of FIG. 8A in a deployed configuration, wherein a first barb and a second barb deflect away from the tissue anchor in opposite directions;

FIG. 9A illustrates a perspective view of an exemplary tissue anchor in a pre-deployed configuration;

FIG. 9B illustrates a side view of the exemplary tissue anchor of FIG. 9A in the pre-deployed configuration;

FIG. 9C illustrates a front view of the exemplary tissue anchor of FIG. 9A in a deployed configuration;

FIG. 9D illustrates a side view of the exemplary tissue anchor of FIG. 9A in a deployed configuration;

FIG. 10A illustrates a perspective view of an exemplary tissue anchor in a pre-deployed configuration;

FIG. 10B illustrates a side view of the exemplary tissue anchor of FIG. 10A in a deployed configuration;

FIG. 11 illustrates an exemplary delivery device;

FIG. 12 illustrates a tendon with an implant fixed thereto using an exemplary tissue anchor;

FIG. 13A illustrates a perspective view of an exemplary tissue anchor;

FIG. 13B illustrates a bottom view of the exemplary tissue anchor of FIG. 13A; and

FIG. 14 illustrates a portion of an exemplary delivery device.

While the disclosure is amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale. Like reference numerals indicate like elements throughout the views. The detailed description and drawings are intended to illustrate but not limit the disclosure. Those skilled in the art will recognize that the various elements described and/or shown may be arranged in various combinations and configurations without departing from the scope of the disclosure.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (i.e., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range (e.g., 1 to 5 includes, 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest dimension measured according to the intended usage. In some instances, an “extent” may generally be measured orthogonally within a plane and/or cross-section, but may be, as will be apparent from the particular context, measured differently-such as, but not limited to, angularly, radially, circumferentially (e.g., along an arc), etc.

It is noted that references in this specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Moreover, such phrases are not necessarily referring to the same embodiment. Additionally, when particular features, structures, and/or characteristics are described in connection with one embodiment, it should be understood that such features, structures, and/or characteristics may also be used in connection with other embodiments whether or not explicitly described unless clearly stated to the contrary. That is, individual elements described herein, even if not explicitly shown in a particular combination, are contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The figures generally illustrate selected components and/or arrangements of medical devices, systems, and/or methods. It should be noted that in any given figure, some features may not be shown, or may be shown schematically, for simplicity. Additional details regarding some elements may be illustrated in other figures in greater detail. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to features or elements may be equally referred to all instances and quantities beyond one of said feature or element. As such, it will be understood that the following discussion may apply equally to any and/or all of the elements for which there are more than one within the medical devices, systems, and/or methods, unless explicitly stated to the contrary.

With its complexity, range of motion and extensive use, a common soft tissue injury is damage to the rotator cuff or rotator cuff tendons. Damage to the rotator cuff is a potentially serious medical condition that may occur during hyperextension, from an acute traumatic tear or from overuse of the joint. Current repair procedures may attempt to alleviate impingement or make room for movement of the tendon to prevent further damage and relieve discomfort. An accepted treatment for rotator cuff tears may include reattaching the torn tendon to the humeral head using sutures or anchors. Additionally, in treating rotator cuff tears, an accepted practice may also include the placement of a scaffold over the repaired tendon to mechanically reinforce the repaired tendon or promote tissue growth for natural repair. The scaffold may be secured to the tendon using one or more, or a plurality of tissue anchors, such as staples or other anchors. Therefore, there is an ongoing need to deliver and adequately secure medical implants during an arthroscopic procedure in order to treat injuries to the rotator cuff, rotator cuff tendons, or other soft tissue or tendon injuries throughout a body. It is noted that although discussion herein is directed to soft tissue repair of the shoulder joint, the devices and associated methods of soft tissue repair may be equally applicable to other joint injuries (e.g., knee, hip, ankle, etc.) and soft tissue repair throughout the patient's body.

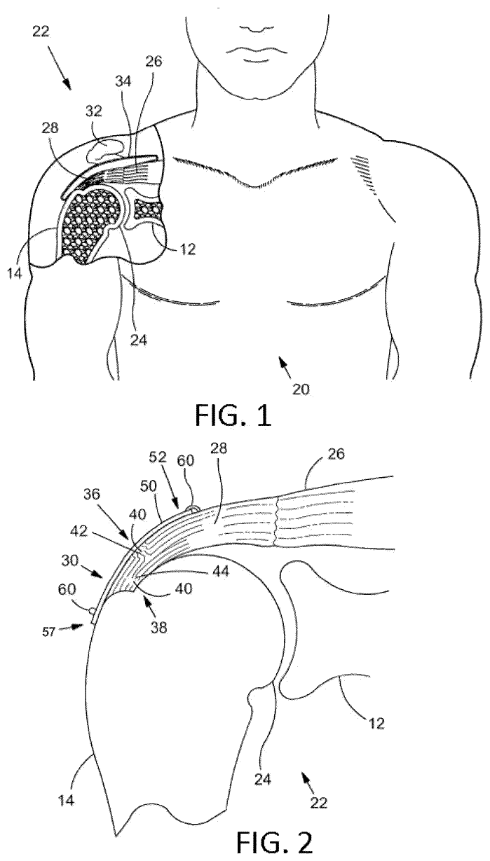

FIG. 1 illustrates an anterior view of a patient 20 including a cross-section of a shoulder 22. The shoulder 22 includes a humerus 14 and a scapula 12. In FIG. 1, a head 24 of the humerus 14 can be seen mating with a glenoid fossa of the scapula 12 at a glenohumeral joint. With reference to FIG. 1, it will be appreciated that the glenoid fossa comprises a shallow depression min the scapula 12. The movement of the humerus 14 relative to the scapula 12 is controlled by a number of muscles including: the deltoid, the supraspinatus, the infraspinatus, the subscapularis, and the teres minor. For purposes of illustration, only a supraspinatus 26 is shown in FIG. 1.

With reference to FIG. 1, it will be appreciated that a distal tendon 28 of the supraspinatus 26 meets the humerus 14 at an insertion point. The scapula 12 of the shoulder 22 includes an acromium 32. In FIG. 1, a subacromial bursa 34 is shown extending between the acromium 32 of the scapula 12 and the head 24 of the humerus 14. In FIG. 1, the subacromial bursa 34 is shown overlaying the supraspinatus 26. The subacromial bursa 34 is one of the hundreds of bursae found the human body. Each bursa comprises a fluid filled sac. The presence of these bursae in in joints throughout the body reduces friction between bodily tissues. Injury and/or infection of the bursa can cause it to become inflamed. This condition is sometimes referred to as bursitis.

The exemplary methods and apparatus described herein may be used to fix tendon repair implants to various target tissues. For example, a tendon repair implant may be fixed to one or more tendons associated with an articulating joint, such as the glenohumeral joint. The tendons to be treated may be torn, partially torn, have internal micro-tears, be untorn, and/or be thinned due to age, injury or overuse. Some of the methods and apparatus of the present application and related devices may provide a beneficial therapeutic effect on a patient experiencing joint pain believed to be caused by full thickness tears, partial thickness tears, and/or internal microtears. A tendon repair implant may be secured to the injured tendon or other soft tissue to provide strength to the injured tendon, carry at least a portion of the load on the tendon and/or promote tissue ingrowth at the injury site.

FIG. 2 illustrates the shoulder 22 including the humerus 14 and the scapula 12 of FIG. 1. In FIG. 2, the head 24 of the humerus 14 is shown mating with the glenoid fossa of the scapula 12 at the glenohumeral joint. The supraspinatus 26 is also shown in FIG. 2. This muscle (along with others) control the movement of the humerus 14 relative to the scapula 12. A distal tendon 28 of the supraspinatus 26 meets the humerus 14 at an insertion point 30.

In the embodiment of FIG. 2, the distal tendon 28 includes a first damaged portion 36. A number of loose tendon fibers 40 in the first damaged portion 36 are visible. The first damaged portion 36 includes a first tear 42 extending partially through the distal tendon 28. The first tear 42 may therefore be referred to as a partial thickness tear. With reference to FIG. 2, it will be appreciated that the first tear 42 begins on the side of the distal tendon 28 facing a subacromial bursa (e.g., subacromial bursa 34) and ends midway through the distal tendon 28. Accordingly, the first tear 42 may be referred to as a bursal side tear. In other instances, the tendon 28 may include a full thickness tear.

With reference to FIG. 2, it will be appreciated that the distal tendon 28 includes a second damaged portion 38 located near the insertion point 30. In the embodiment of FIG. 2, the second damaged portion 38 of the distal tendon 28 has become frayed as indicated by the loose tendon fibers 40. The second damaged portion 38 of the distal tendon 28 includes a second tear 44. It will be appreciated that the second tear 44 begins on the side of the distal tendon 28 facing the humerus 14. Accordingly, the second damaged portion 38 may be referred to as an articular side tear.

A shown in FIG. 2, an implant 50 (e.g., a sheet-like implant) has been placed over the bursal side of the distal tendon 28. The implant 50 may be formed of a biologic material, such as a mesh of collagen fibers or other biocompatible material. It will be appreciated that the implant 50 extends over the insertion point 30, the first tear 42 and the second tear 44. Some useful methods in accordance with this detailed description may include placing a tendon repair implant on the bursal side of a tendon regardless of whether the tears being treated are on the bursal side, articular side or within the tendon. In some cases, the exact location and nature of the tears being treated may be unknown. A tendon repair implant may be applied to the bursal side of a tendon to treat shoulder pain that is most likely caused by one or more partial thickness tears in the tendon. In some cases, delivery of the implant 50 to a target site of a patient may require a physician to create an incision in the patient sufficient to access the target implant site. After creating this “access site,” the physician may insert an implant delivery system through the access site and position the distal end of the implant delivery system adjacent the target implant site. The physician may then manipulate the implant delivery system to deploy an implant out of a delivery sheath (not shown in FIG. 2) or otherwise position the implant adjacent the target implant site. The implant 50 may then be fixed to the distal tendon 28 and to the humerus 14 by a plurality of tissue anchors 60, as described herein in detail.

When positioning the implant 50 adjacent a target site, a clinician may orient the implant 50 such that a proximal portion 51 may be adjacent (e.g., overlaid) on a portion of the humerus 14 (e.g., on the bone), while a distal portion 52 of the implant 50 may overlay the distal tendon 28. Further, once the implant 50 has been placed appropriately, it may be desirable to utilize a stapling instrument (not shown) or other device to insert the plurality of tissue anchors 60 through the implant 50 into the tendon tissue 28 and/or the bone 14. For example, the implant 50 may be anchored to the humeral head using one or more bone anchors (e.g., staples, tacks, etc.) and the implant 50 may be anchored to the tendon 28 using a plurality of tissue anchors (e.g., staples, tacks, etc.) arranged around the periphery of the implant 50.

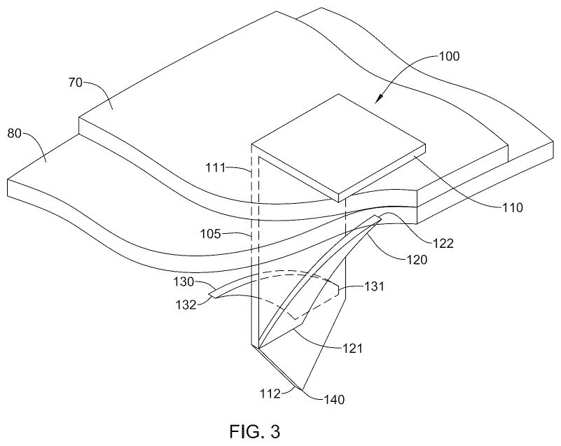

FIG. 3 illustrates a tendon 80 with a tendon repair implant 70 fixed thereto with an exemplary tissue anchor 100. The tendon repair implant 70 may include various sheet-like structures. For example, the sheet-like structure may include a plurality of fibers in which the plurality of fibers may be interlinked or intertwined with one another. In such cases, the sheet-like structure may include a plurality of apertures comprising the interstitial spaces between fibers providing the sheet-like implant 70 with a desired porosity. Various processes may be used to interlink or intertwine the fibers with one another. In some embodiments, the sheet-like structure may include a laminate including multiple layers of film with each layer of film defining a plurality of micro-machined or formed holes. The sheet-like structure of the tendon repair implant 70 may also be formed of a collagen mesh. The sheet-like structure of the tendon repair implant 70 may also include a plurality of electro-spun nanofiber filaments forming a composite sheet. Additionally, the sheet-like structure may comprise a synthetic sponge material that defines a plurality of pores. The sheet-like structure may also comprise a reticulated foam material.

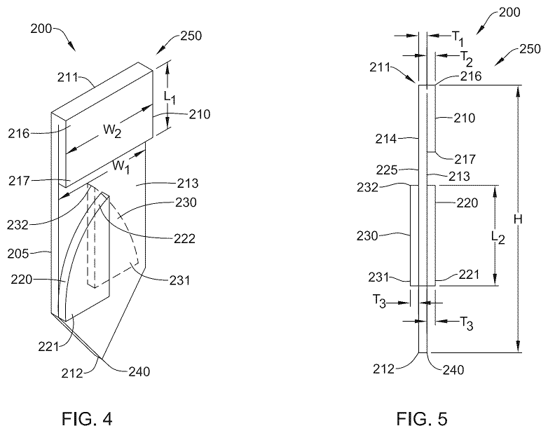

As shown in FIG. 3, the tissue anchor 100 may be used to affix the tendon repair implant 70 to the distal tendon 80. The tissue anchor 100 may include an anchor body 105 having a proximal end 111 and a distal end 112. A tab 110 may be positioned near the proximal end 111. The tab 110 may be movable relative to the anchor body 105 from a first position, in which the tab 110 is in alignment with the anchor body 105 (as shown in FIGS. 4, 5, and 6C), to a second position, in which the tab 110 deflects away from the anchor body 105. The distal end 112 may include a sharp, tissue-penetrating tip such as, for example, a tapered tip or a sharpened distal tip 140. The distal tip 140 may aid the tissue anchor 100 in piercing through the implant 70 and into underlying tissue, such as the distal tendon 80, upon deployment of the tissue anchor 100.

The tissue anchor 100 may further include a first barb 120 positioned near the distal end 112, and a second barb 130 positioned near the distal end 112. The first barb 120 may include a first end or base 121 fixed to the anchor body 105 and a second, free end 122, and the second barb 130 may include a first end or base 131 fixed to the anchor body 105 and a second, free end 132. In some cases, the first barb 120 and the second barb 130 may be positioned on opposite sides of the tissue anchor 100, as shown in FIG. 3. For example, the first barb 120 may be positioned on a first side of the anchor body 105 with the base 121 of the first barb 120 attached to and/or extending from the first side of the anchor body 105, and the second barb 130 may be positioned on a second side of the anchor body 105, opposite the first side of the anchor body 105, with the base 131 of the second barb 130 attached to and/or extending from the second side of the anchor body 105. In some cases, the first barb 120 and the second barb 130 may be positioned on the same side of the tissue anchor 100. While it is shown that the tissue anchor 100 includes two barbs, it may be contemplated that the tissue anchor 100 may include one barb, three barbs, four barbs, or any other suitable number of barbs. In some cases, as shown in FIG. 3, the first barb 120 and the second barb 130 may be movable from a first position, in which the first barb 120 and the second barb 130 are in alignment with the anchor body 105 (as shown in FIGS. 4, 5, and 6C), to a second position, in which the first barb 120 and the second barb 130 deflect away from the anchor body 105. In some cases, the first ends 121, 131 may be attached to the anchor body 105 via heat, laser, or sonic welding, injection molding, adhesive, or any other suitable method of attachment. In other instances, the barbs 120, 130 may be integrally formed with the anchor body 105 and extend therefrom such that the anchor 100 is a monolithic structure including the anchor body 105 and the barbs 120, 130 attached or joined thereto. The second ends 122, 132 may be a free end, thus free to move (e.g., deflect) away from the anchor body 105. In some cases, the first barb 120 and the second barb 130 may deflect away from the anchor body 105 such that the first barb 120 and the second barb 130 may extend across the anchor body 105 wherein the second ends 122, 132 extend away from a side of the anchor body 105 opposite a side of the anchor body 105 that the first ends 121, 131 are attached to. Having the first barb 120 and the second barb 130 deflect (e.g., extend) away from the anchor body 105 may permit the tissue anchor 100 to engage with tissue, such as the distal tendon 80, after the tissue anchor 100 is deployed through the implant 70 and into tissue (e.g., the distal tendon 80).

In some cases, the length and/or deflection configuration of the barbs may vary, or be tuned, to control the depth and/or extend of the barbs as they are forwarded into tissue. Thus, tissue anchors (e.g., tissue anchors 100, 200, 300, 400, 500, 600, 700) may be provided including both barbs that embed less deeply across smaller spans, as well as barbs having a deeper, broader, reach. Thus, although the drawings illustrate deployment of barbs having a uniform configuration, the present disclosure is not so limited.

FIG. 4 illustrates a perspective view of an exemplary tissue anchor 200 in a pre-deployed configuration 250, and FIG. 5 illustrates a side view of the tissue anchor 200 in the pre-deployed configuration 250. The tissue anchor 200 may be an example of the tissue anchor 100. As shown in FIGS. 4 and 5, the tissue anchor 200 may include an anchor body 205 having a front side 213, a back side 214 (shown in FIG. 5), opposing lateral edges extending between the front side 213 and the back side 214, a proximal end 211, and a distal end 212. In some cases, the tissue anchor 200 may include a height H of about 0.250 inches. In some cases, the tissue anchor 200 may include a height H of between about 0.200 inches to about 0.300 inches, about 0.200 inches, about 0.175 inches, about 0.275 inches, about 0.300 inches, or any other suitable height. In some cases, the anchor body 205 may include a width W₁ of about 0.040 inches and a thickness T₁ of about 0.020 inches. In some cases, the anchor body 205 may include a width W₁ of about 0.030 inches to about 0.050 inches, about 0.030 inches, about 0.035 inches, about 0.0375 inches, about 0.045 inches, about 0.050 inches, or any other suitable width. In some cases, the anchor body 205 may include a thickness T₁ of about 0.010 inches to about 0.030 inches, about 0.010 inches, about 0.015 inches, about 0.0175 inches, about 0.025 inches, about 0.030 inches, or any other suitable thickness.

The distal end 212 may include a distal tip 240. The distal tip 240 may be a sharp, tapered tip, and may aid the tissue anchor 200 in piercing through an implant and into tissue upon deployment of the tissue anchor 200. A tab 210 may be positioned near the proximal end 211 of the anchor body 205, and may be movable from a first position, in which the tab 210 is in alignment with the anchor body 205, to a second position, in which the tab 210 deflects away from the anchor body 205. The tab 210 may include a first end 216 which may be attached to the anchor body 205 via heat, laser, sonic welding, injection molding, adhesive, or any other suitable method of attachment. In other instances, the tab 210 may be integrally formed with the anchor body 205 and extend therefrom such that the anchor 200 is a monolithic structure including the anchor body 205 and the tab 210 attached or joined thereto. The tab 210 may further include a second end 217 which may be a free end, thus free to move (e.g., deflect) away from the anchor body 205. When the tissue anchor 200 is in the pre-deployed configuration 250, the tab 210 may fold in toward the anchor body 205 such that the tab 210 may be in the first position in which the tab 210 may abut, face, or be juxtaposed with (e.g., aligned with) the anchor body 205. In the first position, the tab 210 may extend generally parallel to the anchor body 205 and be juxtaposed therewith. In some cases, the first position may be considered to be a first, pre-deployed position, and the second position may be considered to be a second, deployed position. The tab 210 may be biased toward the second, deployed position when unconstrained and not subjected to an external force. Thus, the tab 210 may be configured to automatically transition from the first, pre-deployed position to the second, deployed position when external forces are removed from the tab 210.

In some cases, the tab 210 may include a width W₂ that is the same as the width W₁ of the anchor body 205. In some cases, the tab 210 may include a width W₂ that is not the same as the width W₁ of the anchor body 205. For example, in some cases, the width W₂ of the tab 210 may be about 0.030 inches whereas the width W₁ of the anchor body 205 may be about 0.040 inches. In some cases, the width W₂ of the tab 210 and the width W₁ of the anchor body 205 may both be about 0.040 inches. These are just examples. In some cases, the tab 210 may include a thickness T₂ of about 0.015 inches. In some cases, the tab 210 may include a thickness T₂ of about 0.010 inches to about 0.020 inches, about 0.010 inches, about 0.012 inches, about 0.015 inches, about 0.020 inches, or any other suitable thickness. In some cases, the tab 210 may include a length L₁ of about 0.085 inches. In some cases, the tab 210 may include a length L₁ of about 0.070 inches to about 0.10 inches, about 0.070 inches, about 0.075inches, about 0.080 inches, about 0.090 inches, about 0.10 inches, or any other suitable length. While it is shown that the tab 210 is attached to and/or extends along the front side 213 of the anchor body 205, it may be contemplated that the tab 210 could instead be attached to and/or extend along the back side 214 of the anchor body 205.

As shown in FIGS. 4 and 5, the tissue anchor 200 may include a first barb 220 including a first end or base 221 and a second, free end 222, and a second barb 230 including a first end or base 231 and a second, free end 232 positioned near the distal end 212 of the anchor body 205. The second ends 222, 232 of the barbs 220, 230 may be beveled or otherwise sharpened to enable the barbs 220, 230 to penetrate into tissue, in some instances. The first barb 220 and the second barb 230 may be positioned on opposite sides of the anchor body 205. For example, the first barb 220 may be positioned at and extend from the front side 213 of the anchor body 205, and the second barb 230 may be positioned at and extend from the back side 214 of the anchor body 205. In some cases, each barb 220, 230 may include a length L of at least 0.125 inches. In some cases, the barbs 220, 230 may include a length L of about 0.100 to about 0.200 inches, about 0.100 inches, about 0.125 inches, about 0.150 inches, or any other suitable length. In some cases, the first barb 220 and the second barb 230 may include a thickness T₃ of about 0.015 inches. In some cases, the first barb 220 and the second barb 230 may include a thickness T₃ of about 0.010 inches to about 0.020 inches, about 0.010 inches, about 0.012 inches, about 0.015 inches, about 0.020 inches, or any other suitable thickness. In some cases, the thickness T₃ of the first barb 220 is the same as the thickness T₃ of the second barb 230. In some cases, the thickness T₃ of the first barb 220 may be different than the thickness of the second barb 230. In some cases, the first ends 221, 231 may be attached to the anchor body 205 via heat, laser, or sonic welding, injection molding, adhesive, or any other suitable method of attachment. In other instances, the barbs 220, 230 may be integrally formed with the anchor body 205 and extend therefrom such that the anchor 200 is a monolithic structure including the anchor body 205, the barbs 220, 230, and/or the tab 210 attached or joined thereto. The second ends 222, 232 may be a free end, thus free to move (e.g., deflect) away from the anchor body 205. In some cases, the first barb 220 and the second barb 230 may be movable from a first position, in which the barbs 220, 230 are in alignment with the anchor body 205, to a second position, in which the barbs 220, 230 deflect away from the anchor body 205. When the tissue anchor 200 is in the pre-deployed configuration 250, the barbs 220, 230 may be in the first position in which the barbs 220, 230 may abut, face, or be juxtaposed with (e.g., aligned with) the anchor body 205. In the first position, the barbs 220, 230 may extend generally parallel to the anchor body 205 and be juxtaposed therewith. In some cases, the first position may be considered to be a first, pre-deployed position, and the second position may be considered to be a second, deployed position. The barbs 220, 230 may be biased toward the second, deployed position when unconstrained and not subjected to an external force. Thus, the barbs 220, 230 may be configured to automatically transition from the first, pre-deployed position to the second, deployed position when external forces are removed from the barbs 220, 230. While it is shown that the first barb 220 and the second barb 230 are positioned on opposite s of the anchor body 205, it may be contemplated that the first barb 220 and the second barb 230 may be positioned on the same side of the anchor body 205. In some cases, the tissue anchor 200 may include three barbs, one barb, four barbs, or any other suitable number of barbs.

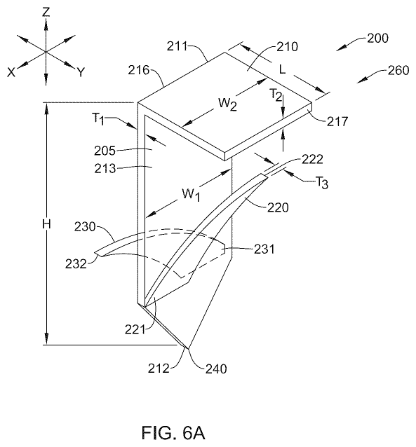

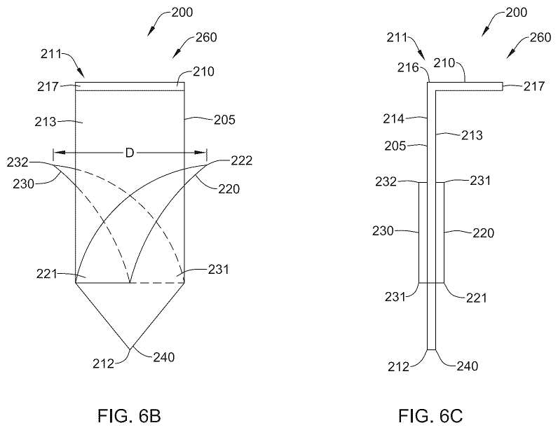

FIGS. 6A-6C illustrate a perspective view, a front view, and a side view, respectively, of the exemplary tissue anchor 200 of FIGS. 4 and 5 in a deployed configuration 260. When the tissue anchor 200 is in the deployed configuration 260, the tab 210 may be in the second position, wherein the first end 216 of the tab 210 remains attached or joined to the anchor body 205, and the second end 217 deflects or extends away from the anchor body 205. The tab 210 may be formed from a shape memory alloy or similar materials with the ability to recover a pre-defined configuration. Such materials may include, but are not limited to, a nickel-titanium alloy, such as Nitinol, and the like. In some cases, the tab 210 may be formed from bioabsorbable or bioerodible materials which may include bioabsorbable polymers (e.g., polylactic acid (PLA), poly-L-lactic acid (PLLA), poly (lactide-co-glycolide) (PLGA), poly-L-lactide-DL-lactic acid (PLDL) or a bioerodible metal (e.g., magnesium alloy). The tab 210 may be biased toward the second position, such that when an applied force is removed from the tab 210, the tab 210 automatically reverts to the second position. For example, the tab 210 may be held within a tissue anchor delivery device in the first, pre-deployed position and upon deployment, the tab 210 may revert to the second, deployed position. The tab 210 may aid in the formation of a secure attachment of an implant (e.g., implant 70) to a tissue (e.g., distal tendon 80) by engaging with a proximal side of the implant, thereby preventing a downward movement of the tissue anchor 200 wherein an entirety of the tissue anchor 200 could become embedded in the tissue, thereby passing completely through the implant.

As shown in FIGS. 6A-6C, the first barb 220 and the second barb 230 may be in the second position in which the first barb 220 and the second barb 230 deflect away from the anchor body 205. The first barb 220 and the second barb 230 may be formed from a shape memory alloy or similar materials with the ability to recover a pre-defined configuration. Such materials may include, but are not limited to, a nickel-titanium alloy, such as Nitinol, and the like. In some cases, the first barb 220 and the second barb 230 may be formed from bioabsorbable or bioerodible materials which may include bioabsorbable polymers (e.g., polylactic acid (PLA), poly-L-lactic acid (PLLA), poly (lactide-co-glycolide) (PLGA), poly-L-lactide-DL-lactic acid (PLDL)) or a bioerodible metal (e.g., magnesium alloy). The first barb 220 and the second barb 230 may be biased toward the second position, such that when an applied force is removed from the tab 210, the first barb 220 and the second barb 230 automatically revert to the second position. For example, the first barb 220 and the second barb 230 may be held within a tissue anchor delivery device in the first, pre-deployed position and upon deployment, the first barb 220 and the second barb 230 may revert to the second, deployed position. In such an arrangement, the barbs 220, 230 are driven into tissue, thereby improving retention of the tissue anchor 200. When the tissue anchor 200 is in the deployed configuration 260, the distance D spanning between the first end 222 of the first barb 220 and the first end 232 of the second barb 230 may be greater than the width W₁ of the anchor body 205, such as a distance of 0.075 inches to about 0.150 inches, or about 0.100 inches. In some cases, the distance D may be about 0.075 inches, about 0.100 inches, about 0.110 inches, about 0.125 inches, or any other suitable distance.

In some cases, when the tab 210 is in the second position and the first barb 220 and the second barb 230 are in the second position, as shown in FIGS. 6A-6C, the tab 210 may deflect away from the anchor body 205 in a first direction, the first barb 220 may deflect away from the anchor body 205 in a second direction, and the second barb 230 may deflect away from the anchor body 205 in a third direction. In some cases, the first and the second directions may be the same direction and the third direction may be different than the first and second directions. In some cases, the first and third directions may be the same direction and the second direction may be different from the first and the third directions. In some cases, the second and third directions may be the same direction and the first direction may be different from the second and third directions. In some cases, the first, second, and third directions may each differ from one another. For example, as shown in FIGS. 6A-6C, with the longitudinal axis of the anchor body 205 extending parallel to and along the Z-Z axis of a coordinate system, the tab 210 may extend (deflect) away from the longitudinal axis of the anchor body 205 (e.g., may be perpendicular to the anchor body 205) in the positive Y direction. The first barb 220 and the second barb 230 may extend (deflect) away from the longitudinal axis of the anchor body 205 such that the second ends 222, 232 extend away from opposite sides or edges of the anchor body 205 in the negative X direction and the positive X direction, respectively.

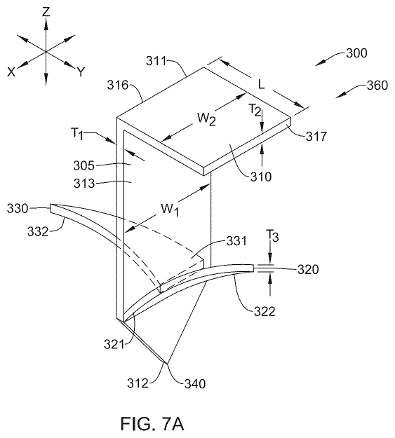

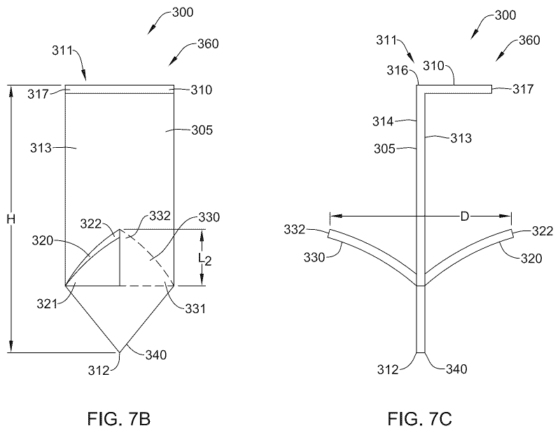

FIG. 7A-7C illustrates a perspective view, a front view, and a side view, respectively, of an exemplary tissue anchor 300 in a deployed configuration 360. The tissue anchor 300 may be an example of the tissue anchor 100. The tissue anchor 300 may include an anchor body 305 having a front side 313, a back side 314, opposing lateral edges extending between the front side 313 and the back side 314, a proximal end 311, and a distal end 312. In some cases, the tissue anchor 300 may include a height H of about 0.250 inches. In some cases, the tissue anchor 300 may include a height H of between about 0.200 inches to about 0.300 inches, about 0.200 inches, about 0.175 inches, about 0.275 inches, about 0.300 inches, or any other suitable height. In some cases, the anchor body 305 may include a width W₁ of about 0.040 inches and a thickness T₁ of about 0.020 inches. In some cases, the anchor body 305 may include a width W₁ of about 0.030 inches to about 0.050 inches, about 0.030 inches, about 0.035 inches, about 0.0375 inches, about 0.045 inches, about 0.050 inches, or any other suitable width. In some cases, the anchor body 305 may include a thickness T₁ of about 0.010 inches to about 0.030 inches, about 0.010 inches, about 0.015 inches, about 0.0175 inches, about 0.025 inches, about 0.030 inches, or any other suitable thickness.

The distal end 312 may include a distal tip 340. The distal tip 340 may be a sharp, tapered tip, and may aid the tissue anchor 300 in piercing through an implant and into tissue upon deployment of the tissue anchor 300. A tab 310 may be positioned near the proximal end 311 of the anchor body 305, and may be movable from a first position, in which the tab 310 is in alignment with the anchor body 305, to a second position, in which the tab 310 deflects away from the anchor body 305. The tab 310 may include a first end 316 which may be attached to the anchor body 305 via heat, laser, sonic welding, injection molding, adhesive, or any other suitable method of attachment. In other instances, the tab 310 may be integrally formed with the anchor body 305 and extend therefrom such that the anchor 300 is a monolithic structure including the anchor body 305 and the tab 310 attached or joined thereto. The tab 310 may further include a second end 317 which may be a free end, thus free to move (e.g., deflect) away from the anchor body 305. When the tissue anchor 300 is in a pre-deployed configuration (e.g., pre-deployed configuration 250), the tab 310 may fold in toward the anchor body 305 such that the tab 310 may be in the first position in which the tab 310 may abut, face, or be juxtaposed with (e.g., aligned with) the anchor body 305. In the first position, the tab 310 may extend generally parallel to the anchor body 305 and be juxtaposed therewith. In some cases, the first position may be considered to be a first, pre-deployed position, and the second position may be considered to be a second, deployed position. The tab 310 may be biased toward the second, deployed position when unconstrained and not subjected to an external force. Thus, the tab 310 may be configured to automatically transition from the first, pre-deployed position to the second, deployed position when external forces are removed from the tab 310.

In some cases, the tab 310 may include a width W₂ that is the same as the width W₁ of the anchor body 305. In some cases, the tab 310 may include a width W₂ that is not the same as the width W₁ of the anchor body 305. For example, in some cases, the width W₂ of the tab 310 may be about 0.030 inches whereas the width W₁ of the anchor body 305 may be about 0.040 inches. In some cases, the width W₂ of the tab 310 and the width W₁ of the anchor body 305 may both be about 0.040 inches. These are just examples. In some cases, the tab 310 may include a thickness T₂ of about 0.015 inches. In some cases, the tab 310 may include a thickness T₂ of about 0.010 inches to about 0.020 inches, about 0.010 inches, about 0.012 inches, about 0.015 inches, about 0.020 inches, or any other suitable thickness. In some cases, the tab 310 may include a length L₁ of about 0.085 inches. In some cases, the tab 310 may include a length L₁ of about 0.070 inches to about 0.10 inches, about 0.070 inches, about 0.075inches, about 0.080 inches, about 0.090 inches, about 0.10 inches, or any other suitable length. While it is shown that the tab 310 is attached to and/or extends along the front side 313 of the anchor body 305, it may be contemplated that the tab 310 could instead be attached to and/or extend along the back side 314 of the anchor body 305. When the tissue anchor 300 is in the deployed configuration 360, the tab 310 may be in the second position, wherein the first end 316 of the tab 310 remains attached or joined to the anchor body 305, and the second end 317 deflects or extends away from the anchor body 305. The tab 310 may be formed from a shape memory alloy or similar materials with the ability to recover a pre-defined configuration. Such materials may include, but are not limited to, a nickel-titanium alloy, such as Nitinol, and the like. In some cases, the tab 310 may be formed from bioabsorbable or bioerodible materials which may include bioabsorbable polymers (e.g., polylactic acid (PLA), poly-L-lactic acid (PLLA), poly (lactide-co-glycolide) (PLGA), poly-L-lactide-DL-lactic acid (PLDL)) or a bioerodible metal (e.g., magnesium alloy). The tab 310 may be biased toward the second position, such that when an applied force is removed from the tab 310, the tab 310 automatically reverts to the second position. For example, the tab 310 may be held within a tissue anchor delivery device in the first, pre-deployed position and upon deployment, the tab 310 may revert to the second, deployed position. The tab 310 may aid in the formation of a secure attachment of an implant (e.g., implant 70) to a tissue (e.g., distal tendon 80) by engaging with a proximal side of the implant, thereby preventing a downward movement of the tissue anchor 300 wherein an entirety of the tissue anchor 300 could become embedded in the tissue, thereby passing completely through the implant.

The tissue anchor 300 may include a first barb 320 including a first end 321 or base and a second, free end 322, and a second barb 330 including a first end 331 or base and a second, free end 332 positioned near the distal end 312 of the anchor body 305. In some cases, the first ends 321, 331 may be attached to the anchor body 305 via heat, laser, or sonic welding, injection molding, adhesive, or any other suitable method of attachment. In other instances, the barbs 320, 330 may be integrally formed with the anchor body 305 and extend therefrom such that the anchor 300 is a monolithic structure including the anchor body 305, the barbs 320, 330, and/or the tab 310 attached or joined thereto. The second ends 322, 332 may be a free end, thus free to move (e.g., deflect) away from the anchor body 305. In some cases, the first barb 320 and the second barb 330 may be movable from a first position, in which the barbs 320, 330 are in alignment with the anchor body 305, to a second position, in which the barbs 320, 330 deflect away from the anchor body 305. The second ends 322, 332 of the barbs 320, 330 may be beveled or otherwise sharpened to enable the barbs 320, 330 to penetrate into tissue, in some instances. The first barb 320 and the second barb 330 may be positioned on opposite sides of the anchor body 305. For example, the first barb 320 may be positioned at and extend from the front side 313 of the anchor body 305, and the second barb 330 may be positioned at and extend from the back side 314 of the anchor body 305. In some cases, each barb 320, 330 may include a length L of at least 0.125 inches. In some cases, the barbs 320, 330 may include a length L of about 0.100 to about 0.200 inches, about 0.100 inches, about 0.125 inches, about 0.150 inches, or any other suitable length. In some cases, the first barb 320 and the second barb 330 may include a thickness T₃ of about 0.015 inches. In some cases, the first barb 320 and the second barb 330 may include a thickness T₃ of about 0.010 inches to about 0.020 inches, about 0.010 inches, about 0.012 inches, about 0.015 inches, about 0.020 inches, or any other suitable thickness. In some cases, the thickness T₃ of the first barb 320 is the same as the thickness T₃ of the second barb 330. In some cases, the thickness T₃ of the first barb 320 may be different than the thickness of the second barb 330.

When the tissue anchor 300 is in the pre-deployed configuration, the barbs 320, 330 may be in the first position in which the barbs 320, 330 may abut, face, or be juxtaposed with (e.g., aligned with) the anchor body 305. In the first position, the barbs 320, 330 may extend generally parallel to the anchor body 205 and be juxtaposed therewith. In some cases, the first position may be considered to be a first, pre-deployed position, and the second position may be considered to be a second, deployed position. The barbs 320, 330 may be biased toward the second, deployed position when unconstrained and not subjected to an external force. Thus, the barbs 320, 330 may be configured to automatically transition from the first, pre-deployed position to the second, deployed position when external forces are removed from the barbs 320, 330. While it is shown that the first barb 320 and the second barb 330 are positioned on opposite sides of the anchor body 305 (e.g., the front side 313 and the back side 314, respectively), it may be contemplated that the first barb 320 and the second barb 330 may be positioned on the same side of the anchor body 305. In some cases, the tissue anchor 300 may include three barbs, one barb, four barbs, or any other suitable number of barbs.

As shown in FIGS. 7A-7C, the first barb 320 and the second barb 330 may be in the second position in which the first barb 320 and the second barb 330 deflect away from the anchor body 305. The first barb 320 and the second barb 330 may be formed from a shape memory alloy or similar materials with the ability to recover a pre-defined configuration. Such materials may include, but are not limited to, a nickel-titanium alloy, such as Nitinol, and the like. In some cases, the first barb 320 and the second barb 330 may be formed from bioabsorbable or bioerodible materials which may include bioabsorbable polymers (e.g., polylactic acid (PLA), poly-L-lactic acid (PLLA), poly (lactide-co-glycolide) (PLGA), poly-L-lactide-DL-lactic acid (PLDL)) or a bioerodible metal (e.g., magnesium alloy). The first barb 320 and the second barb 330 may be biased toward the second position, such that when an applied force is removed from the tab 310, the first barb 320 and the second barb 330 automatically revert to the second position. For example, the first barb 320 and the second barb 330 may be held within a tissue anchor delivery device in the first, pre-deployed position and upon deployment, the first barb 320 and the second barb 330 may revert to the second, deployed position. In such an arrangement, the barbs 320, 330 are driven into tissue, thereby improving retention of the tissue anchor 300. When the tissue anchor 300 is in the deployed configuration 360, the distance D spanning between the first end 322 of the first barb 320 and the first end 332 of the second barb 330 may be greater than the width W₁ of the anchor body 205, such as a distance of 0.075 inches to about 0.150 inches, or about 0.100 inches. In some cases, the distance D may be about 0.075 inches, about 0.100 inches, about 0.110 inches, about 0.125 inches, or any other suitable distance.

In some cases, when the tab 310 is in the second position and the first barb 320 and the second barb 330 are in the second position, as shown in FIGS. 7A-7C, the tab 310 may deflect away from the anchor body 305 in a first direction, the first barb 320 may deflect away from the anchor body 305 in a second direction, and the second barb 330 may deflect away from the anchor body 305 in a third direction. In some cases, the first and the second directions may be the same direction and the third direction may be different than the first and second directions. In some cases, the first and third directions may be the same direction and the second direction may be different from the first and the third directions. In some cases, the second and third directions may be the same direction and the first direction may be different from the second and third directions. In some cases, the first, second, and third directions differ from one another. For example, as shown in FIGS. 7A-7C, with the longitudinal axis of the anchor body 305 extending parallel to and along the Z-Z axis of a coordinate system, the tab 310 may extend (deflect) away from the longitudinal axis of the anchor body 305 (e.g., may be perpendicular to the anchor body 305) in the positive Y direction. The first barb 320 and the second barb 330 may extend (deflect) away from the longitudinal axis of the anchor body 305 such that the second end 322 of the first barb 320 deflects away from the front side 313 of the anchor body 305 in the positive Y direction and the second end 332 of the second barb 330 deflects away from the back side 314 of the anchor body 305 in the negative Y direction.

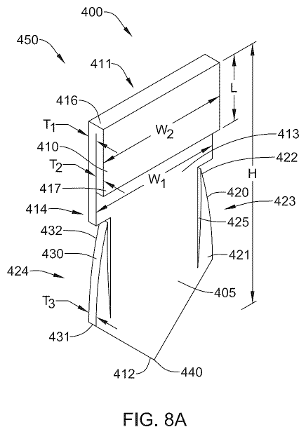

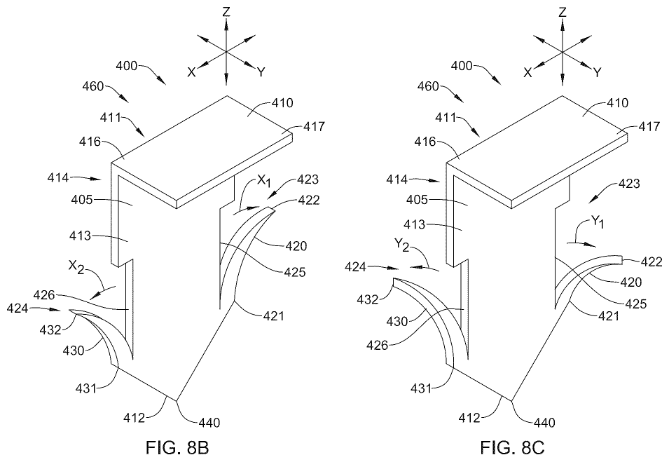

FIG. 8A illustrates a perspective view of an exemplary tissue anchor 400 in a pre-deployed configuration 450, FIG. 8B illustrates the tissue anchor 400 in a deployed configuration 460, and FIG. 8C illustrates the tissue anchor in an alternative deployed configuration 470. As shown in FIGS. 8A-8C, the tissue anchor 400 may include an anchor body 405 having a front side 413, a back side 414, a first lateral side 423 extending between the front side 413 and the back side 414, a second lateral side 424 extending between the front side 413 and the back side 414, a proximal end 411, and a distal end 412. In some cases, the tissue anchor 400 may include a height H of about 0.250 inches. In some cases, the tissue anchor 400 may include a height H of between about 0.200 inches to about 0.300 inches, about 0.200 inches, about 0.175 inches, about 0.275 inches, about 0.300 inches, or any other suitable height. In some cases, the anchor body 405 may include a width W₁ of about 0.040 inches and a thickness T₁ of about 0.020 inches. In some cases, the anchor body 405 may include a width W₁ of about 0.030 inches to about 0.050 inches, about 0.030 inches, about 0.035 inches, about 0.0375 inches, about 0.045 inches, about 0.050 inches, or any other suitable width. In some cases, the anchor body 405 may include a thickness T₁ of about 0.010 inches to about 0.030 inches, about 0.010 inches, about 0.015 inches, about 0.0175 inches, about 0.025 inches, about 0.030 inches, or any other suitable thickness.

The distal end 412 may include a distal tip 440. The distal tip 440 may be a sharp, tapered tip, and may aid the tissue anchor 400 in piercing through an implant and into tissue upon deployment of the tissue anchor 400. A tab 410 may be positioned near the proximal end 411 of the anchor body 405, and may be movable from a first position, in which the tab 410 is in alignment with the anchor body 405, to a second position, in which the tab 410 deflects away from the anchor body 405. The tab 410 may include a first end 416 which may be attached to the anchor body 405 via heat, laser, sonic welding, injection molding, adhesive, or any other suitable method of attachment. In other instances, the tab 410 may be integrally formed with the anchor body 405 and extend therefrom such that the anchor 400 is a monolithic structure including the anchor body 405 and the tab 410 attached or joined thereto. The tab 410 may further include a second end 417 which may be a free end, thus free to move (e.g., deflect) away from the anchor body 405. When the tissue anchor 400 is in a pre-deployed configuration 450, the tab 410 may fold in toward the anchor body 405 such that the tab 410 may be in the first position in which the tab 410 may abut, face, or be juxtaposed with (e.g., aligned with) the anchor body 405. In the first position, the tab 410 may extend generally parallel to the anchor body 305 and be juxtaposed therewith. In some cases, the first position may be considered to be a first, pre-deployed position, and the second position may be considered to be a second, deployed position. The tab 410 may be biased toward the second, deployed position when unconstrained and not subjected to an external force. Thus, the tab 410 may be configured to automatically transition from the first, pre-deployed position to the second, deployed position when external forces are removed from the tab 410.

In some cases, the tab 410 may include a width W₂ that is the same as the width W₁ of the anchor body 405. In some cases, the tab 410 may include a width W₂ that is not the same as the width W₁ of the anchor body 405. For example, in some cases, the width W₂ of the tab 410 may be about 0.030 inches whereas the width W₁ of the anchor body 405 may be about 0.040 inches. In some cases, the width W₂ of the tab 410 and the width W₁ of the anchor body 405 may both be about 0.040 inches. These are just examples. In some cases, the tab 410 may include a thickness T₂ of about 0.015 inches. In some cases, the tab 410 may include a thickness T₂ of about 0.010 inches to about 0.020 inches, about 0.010 inches, about 0.012 inches, about 0.015 inches, about 0.020 inches, or any other suitable thickness. In some cases, the tab 410 may include a length L₁ of about 0.085 inches. In some cases, the tab 410 may include a length L₁ of about 0.070 inches to about 0.10 inches, about 0.070 inches, about 0.075inches, about 0.080 inches, about 0.090 inches, about 0.10 inches, or any other suitable length. While it is shown that the tab 410 is attached to and/or extends along the front side 413 of the anchor body 405, it may be contemplated that the tab 410 could instead be attached to and/or extend along the back side 414 of the anchor body 405.

The tissue anchor 400 may include a first barb 420 including a first end 421 or base and a second, free end 422, and a second barb 430 including a first end 431 or base and a second, free end 432 positioned near the distal end 412 of the anchor body 405. In some cases, the first ends 421, 431 may be attached to the anchor body 405 via heat, laser, or sonic welding, injection molding, adhesive, or any other suitable method of attachment. In other instances, the barbs 420, 430 may be integrally formed with the anchor body 405 and extend therefrom such that the anchor 400 is a monolithic structure including the anchor body 405, the barbs 420, 430, and/or the tab 410 attached or joined thereto. The second ends 422, 432 may be a free end, thus free to move (e.g., deflect) away from the anchor body 405. In some cases, the first barb 420 and the second barb 430 may be movable from a first position, in which the barbs 420, 430 are in alignment with the anchor body 405, to a second position, in which the barbs 420, 430 deflect away from a longitudinal axis the anchor body 405. The second ends 422, 432 of the barbs 420, 430 may be beveled or otherwise sharpened to enable the barbs 420, 430 to penetrate into tissue, in some instances. The first barb 420 and the second barb 430 may be positioned on opposite sides of the anchor body 405. For example, the first barb 420 may be positioned at the first lateral side 423 of the anchor body 405, and the second barb 430 may be positioned at the second lateral side 424 of the anchor body 405. In some cases, the first and second barbs 420, 430 may include dimensions (e.g., length L, thickness T₃, distance D) similar to those described above with respect to FIGS. 7A-7C.

As shown in FIGS. 8A-8C, the anchor body 405 may include a first cut-out 425 within the first lateral side 423 and a second cut-out 426 within the second lateral side 424. The cut-outs 425, 426 may each include a size configured to accommodate the first barb 420 and the second barb 430, respectively. Thus, when the tissue anchor 400 is in the pre-deployed configuration 450, the anchor body 405, the first barb 420, and the second barb 430 align, such that a surface of the first barb 420 is juxtaposed with the first lateral side 423 of the anchor body 405, and a surface of the second barb 430 is juxtaposed with the second lateral side 424 of the anchor body 405, as shown in FIG. 8A. Thus, the first barb 420 and the second barb may lay in the same plane as the anchor body 405 in the pre-deployed configuration 450.

As shown in FIGS. 8B-8C, the first barb 420 and the second barb 430 may be in the second position, which may be considered a deployed configuration, in which the first barb 420 and the second barb 430 deflect away from the anchor body 405. The first barb 420 and the second barb 430 may be biased toward the second position when unconstrained and not subjected to an external force, such that when an applied force is removed from the tab 410, the first barb 420 and the second barb 430 automatically revert to the second position. For example, the first barb 420 and the second barb 430 may be held within a tissue anchor delivery device in the first, pre-deployed position and upon deployment, the first barb 420 and the second barb 430 may automatically revert to the second, deployed position. In such an arrangement, the barbs 420, 430 are driven into tissue, thereby improving retention of the tissue anchor 400.

In some cases, when the tab 410 is in the second position and the first barb 420 and the second barb 430 are in the second position, as shown in FIGS. 8B-8C, the tab 410 may deflect away from the anchor body 405 in a first direction, the first barb 420 may deflect away from the anchor body 405 in a second direction, and the second barb 430 may deflect away from the anchor body 405 in a third direction. In some cases, the first and the second directions may be the same direction and the third direction may be different than the first and second directions. In some cases, the first and third directions may be the same direction and the second direction may be different from the first and the third directions. In some cases, the second and third directions may be the same direction and the first direction may be different from the second and third directions. In some cases, the first, second, and third directions differ from one another. For example, as shown in FIG. 8B, when the tissue anchor 400 is in the deployed configuration 460 with the longitudinal axis of the anchor body 405 extending parallel to and along the Z-Z axis of a coordinate system, the tab 410 may extend (deflect) away from the longitudinal axis of the anchor body 405 (e.g., may be perpendicular to the anchor body 405) in the positive Y direction. The first barb 420 may extend (deflect) away from the anchor body 405 in a first lateral direction, as indicated by arrow X₁ in the negative X direction, and the second barb 430 may extend (deflect) away from the anchor body 405 in a second lateral direction, as indicated by arrow X₂ in the positive X direction. In such cases, the first end 422 of the first barb 420 deflects laterally outward and away from the first lateral side 423 of the anchor body 405 and the first end 432 of the second barb 430 deflects laterally outward and away from the second lateral side 424 of the anchor body 405, such that the anchor body 405, the first barb 420, and the second barb 430 remain in the same plane.

As shown in FIG. 8C, the tissue anchor 400 is in the alternative deployed configuration 470. While in the deployed configuration 470 with the longitudinal axis of the anchor body 405 extending parallel to and along the Z-Z axis of a coordinate system, the tab 410 may extend (deflect) away from the longitudinal axis of the anchor body 405 (e.g., may be perpendicular to the anchor body 405) in the positive Y direction. The first barb 420 may extend (deflect) away from the anchor body 405 in a first anterior direction, as indicated by arrow Y₁ in the positive Y direction, and the second barb 430 may extend (deflect) away from the anchor body 405 in a second posterior direction, opposite the first anterior direction, as indicated by arrow Y₂ in the negative Y direction, such that the first end 422 of the first barb 420 deflects away from the front side 413 of the anchor body 405 in a forward direction, and the first end 432 of the second barb 430 deflects away from the back side 414 of the anchor body 405 in a posterior direction.

In some cases, the tab 410, the first barb 420, and the second barb 430 may be formed from a shape memory alloy or similar materials with the ability to recover a pre-defined configuration, as described above. In some cases, the tab 410, the first barb 420, and the second barb 430 may be formed from bioabsorbable or bioerodible materials, as described above. The tab 410, the first barb 420, and the second barb 430 may be biased toward the second position, such that when an applied force is removed from the tab 410, the first barb 420, and the second barb 430 automatically revert to the second position.

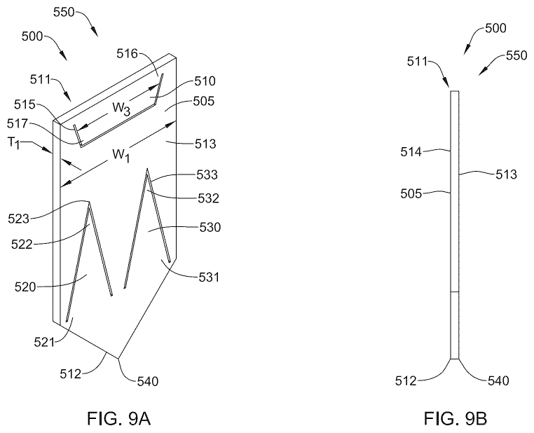

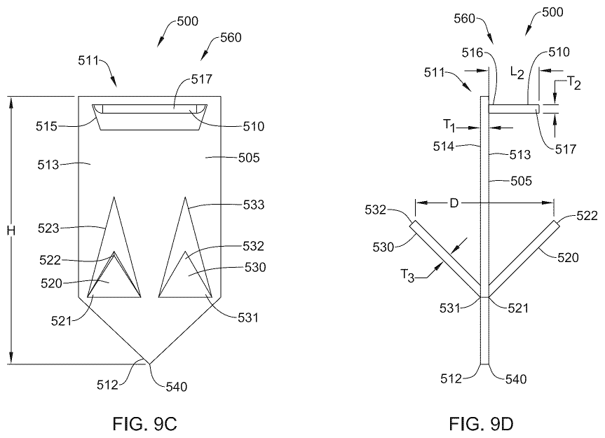

FIGS. 9A-9D illustrate an exemplary tissue anchor 500. FIGS. 9A-9B illustrate a perspective view and a side view, respectively, of the tissue anchor 500 in a first, pre-deployed configuration 550, and FIGS. 9C-9D illustrate a front view and a side view, respectively, of the tissue anchor 500 in a second, deployed configuration 560. As shown in FIGS. 9A-9D, the tissue anchor 500 may include an anchor body 505 having a front side 513, a back side 514, a first lateral edge extending between the front side 513 and the back side 514, an opposite second lateral edge extending between the front side 513 and the back side 514, a proximal end 511, and a distal end 512. In some cases, the tissue anchor 500 may include a height H of about 0.250 inches. In some cases, the tissue anchor 500 may include a height H of between about 0.200 inches to about 0.300 inches, about 0.200 inches, about 0.175 inches, about 0.275 inches, about 0.300 inches, or any other suitable height. In some cases, the anchor body 505 may include a width W₁ of about 0.040 inches and a thickness T₁ of about 0.020 inches. In some cases, the anchor body 505 may include a width W₁ of about 0.030 inches to about 0.050 inches, about 0.030 inches, about 0.035 inches, about 0.0375 inches, about 0.045 inches, about 0.050 inches, or any other suitable width. In some cases, the anchor body 505 may include a thickness T₁ of about 0.010 inches to about 0.030 inches, about 0.010 inches, about 0.015 inches, about 0.0175 inches, about 0.025 inches, about 0.030 inches, or any other suitable thickness.

The distal end 512 may include a distal tip 540. The distal tip 540 may be a sharp, tapered tip, and may aid the tissue anchor 500 in piercing through an implant and into tissue upon deployment of the tissue anchor 500. A tab 510 may be positioned near the proximal end 511 of the anchor body 505, and may be movable from a first position, in which the tab 510 is in alignment with the anchor body 505, to a second position, in which the tab 510 deflects away from the anchor body 505. In the embodiment shown in FIGS. 9A-9D, the tissue anchor 500 may be a monolithic structure in which the tab 510 may include a first, base end 516 which may be attached or joined to the anchor body 505, and a second end 517 which may be a free end, thus free to move (e.g., deflect) away from the anchor body 505. The tab 510 may be cut out of the anchor body 505 such that the anchor body 505 includes a tab opening 515, wherein the tab 510 is located within a perimeter of the tab opening 515 and lays in the same plane as the anchor body 505 when in the first position, and the second end 517 of the tab 510 may extend outward from the tab opening 515 when in the second position. In some cases, the first position may be considered to be a first, pre-deployed position, and the second position may be considered to be a second, deployed position. The tab 510 may include a width W₃ that is not the same as the width W₁ of the anchor body 505. For example, in some cases, the width W₃ of the tab 510 may be about 0.030 inches whereas the width W₁ of the anchor body 505 may be about 0.040 inches. The tab 510 may be biased toward the second, deployed position when unconstrained and not subjected to an external force. Thus, the tab 510 may be configured to automatically transition from the first, pre-deployed position to the second, deployed position when external forces are removed from the tab 510.

The monolithic structure of the tissue anchor 500 may also include a first barb 520 including a first, base end 521 and a second, free end 522, and a second barb 530 including a first, base end 531 and a second, free end 532 positioned near the distal end 512 of the anchor body 505. The first ends 521, 531 may be joined to the anchor body 505, and the second ends 532, 533 may be free to move (e.g., deflect) away from the anchor body 505. The barbs 520, 530 may be cut out of the anchor body 505 such that the anchor body 505 includes a first barb opening 523 and a second barb opening 533, wherein the first barb 520 and the second barb 530 are located within a perimeter of the first barb opening 523 and the second barb opening 533, respectively, when in the first position, and the second ends 522, 532 of the barbs 520, 530 may extend outward from the openings 523, 533 when in the second position. In some cases, the first position may be considered to be a first, pre-deployed position, and the second position may be considered to be a second, deployed position.

In some cases, the first barb 520 and the second barb 530 may be movable from a first position, in which the barbs 520, 530 are in alignment with the anchor body 505 (e.g., extend along the same plane as the anchor body 505), as shown in FIGS, 9A-9B, to a second position, in which the barbs 520, 530 deflect away from a longitudinal axis the anchor body 505, as shown in FIGS. 9C-9D. The barbs 520, 530 may be biased toward the second, deployed position when unconstrained and not subjected to an external force. Thus, the barbs 520, 530 may be configured to automatically transition from the first, pre-deployed position to the second, deployed position when external forces are removed from the barbs 520, 530. The second ends 522, 532 of the barbs 520, 530 may be beveled or otherwise sharpened to enable the barbs 520, 530 to penetrate into tissue. The first barb 520 and the second barb 530 may extend in opposite directions, away from the longitudinal axis of the anchor body 505 in the deployed configuration, in some instances. For example, as shown in FIGS. 9C-9D, the first barb 520 may extend outward of and away from the front side 513 of the anchor body 505, and the second barb 530 may extend outward of and away from the back side 514 of the anchor body 505, such that a distance D between the first ends 522, 532 may be a distance of about 0.075 inches to about 0.125 inches, for example. In some cases, the distance D may include be about 0.075 inches, about 0.100 inches, about 0.125 inches, about 0.110 inches, about 0.090 inches, or any other suitable distance. In other instances, the barbs 520, 530 may extend from the same side (e.g., front side 513 or back side 514) of the anchor body 505 in the deployed configuration.

In some cases, the first and second barbs 520, 530 may include dimensions (e.g., length L, thickness T₃) similar to those described above with respect to FIGS. 7A-7C. In some cases, the tab 510, the first barb 520, and the second barb 530 may be formed monolithically with the anchor body 505 from a shape memory alloy or similar materials with the ability to recover a pre-defined configuration, as described above. In some cases, the tab 510, the first barb 520, and the second barb 530 may be formed from bioabsorbable or bioerodible materials, as described above. The tab 510, the first bard 520, and the second barb 530 may be heat set in the second, deployed configuration. Thus, the tab 510, the first barb 520, and the second barb 530 may be biased toward the second position, such that when an applied force is removed from the anchor 500, the tab 510, the first barb 520, and the second barb 530 automatically revert to the second position.

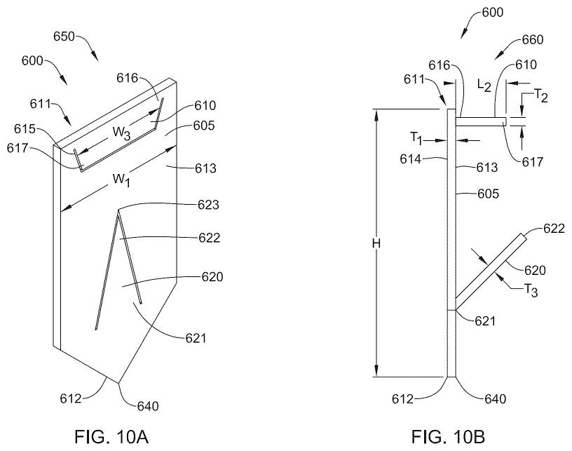

FIGS. 10A-10B illustrate an exemplary tissue anchor 600. FIG. 10A illustrates the tissue anchor 600 in a first, pre-deployed configuration 650, and FIG. 10B illustrates the tissue anchor 600 in a second, deployed configuration 660. The tissue anchor 600 may be an alternative example of the tissue anchor 500 described above. The tissue anchor 600 may include an anchor body 605 having a front side 613, a back side 614, a first lateral edge extending between the front side 613 and the back side 614, an opposite second lateral edge extending between the front side 613 and the back side 614, a proximal end 611, and a distal end 612. In some cases, the tissue anchor 600 may include a height H of about 0.250 inches. In some cases, the tissue anchor 600 may include a height H of between about 0.200 inches to about 0.300 inches, about 0.200 inches, about 0.175 inches, about 0.275 inches, about 0.300 inches, or any other suitable height.

In some cases, the anchor body 605 may include a width W₁ of about 0.040 inches and a thickness T₁ of about 0.020 inches. In some cases, the anchor body 605 may include a width W₁of about 0.030 inches to about 0.050 inches, about 0.030 inches, about 0.035 inches, about 0.0375 inches, about 0.045 inches, about 0.050 inches, or any other suitable width. In some cases, the anchor body 605 may include a thickness T₁ of about 0.010 inches to about 0.030 inches, about 0.010 inches, about 0.015 inches, about 0.0175 inches, about 0.025 inches, about 0.030 inches, or any other suitable thickness.

The distal end 612 may include a distal tip 640. The distal tip 640 may be a sharp, tapered tip, and may aid the tissue anchor 600 in piercing through an implant and into tissue upon deployment of the tissue anchor 600. A tab 610 may be positioned near the proximal end 611 of the anchor body 605, and may be movable from a first position, in which the tab 610 is in alignment with the anchor body 605, to a second position, in which the tab 610 deflects away from the anchor body 605. In the embodiment shown in FIGS. 10A-10B, the tissue anchor 600 may be a monolithic structure in which the tab 610 may include a first, base end 616 which may be attached or joined to the anchor body 605, and a second end 617 which may be a free end, thus free to move (e.g., deflect) away from the anchor body 605. The tab 610 may be cut out of the anchor body 605 such that the anchor body 605 includes a tab opening 615, wherein the tab 610 is located within a perimeter of the tab opening 615 and lays in the same plane as the anchor body 505 when in the first position, and the second end 617 of the tab 610 may extend outward from the tab opening 615 when in the second position. In some cases, the first position may be considered to be a first, pre-deployed position, and the second position may be considered to be a second, deployed position. The tab 610 may include a width W₃ that is not the same as the width W₁ of the anchor body 605. For example, in some cases, the width W₃ of the tab 610 may be about 0.030 inches whereas the width W₁ of the anchor body 605 may be about 0.040 inches. The tab 610 may be biased toward the second, deployed position when unconstrained and not subjected to an external force. Thus, the tab 610 may be configured to automatically transition from the first, pre-deployed position to the second, deployed position when external forces are removed from the tab 610.

The monolithic structure of the tissue anchor 600 may also include a first barb 620 including a first, base end 621 and a second, free end 622 positioned near the distal end 612 of the anchor body 605. The first end 521 may be joined to the anchor body 605, and the second end 532 may be free to move (e.g., deflect) away from the anchor body 605. The first barb 620 may be cut out of the anchor body 605 such that the anchor body 605 includes a first barb opening 623, wherein the first barb 620 is located within a perimeter of the first barb opening 623, when in the first position, and the second end 622 of the barb 620 may extend outward from the opening 623 when in the second position. In some cases, the first position may be considered to be a first, pre-deployed position, and the second position may be considered to be a second, deployed position.

In some cases, the first barb 620 may be movable from a first position, in which the barb 620 is in alignment with the anchor body 605 (e.g., extends along the same plane as the anchor body 605), as shown in FIG. 10A, to a second position, in which the first barb 520 deflects away from a longitudinal axis the anchor body 605, as shown in FIG. 10B. The barb 620 may be biased toward the second, deployed position when unconstrained and not subjected to an external force. Thus, the barb 620 may be configured to automatically transition from the first, pre-deployed position to the second, deployed position when external forces are removed from the barb 620. The second end 622 of the barb 620 may be beveled or otherwise sharpened to enable the barb 620 to penetrate into tissue.

In some cases, the first barb 620 may include dimensions (e.g., length L, thickness T₃) similar to those described above with respect to FIGS. 7A-7C. In some cases, the tab 610 and the first barb 620 may be formed monolithically with the anchor body 605 from a shape memory alloy or similar materials with the ability to recover a pre-defined configuration, as described above. In some cases, the tab 610, the first barb 620, and the second barb 630 may be formed from bioabsorbable or bioerodible materials, as described above. The tab 610 and the barb 620 may be heat set in the second, deployed configuration. Thus the tab 610 and the first barb 620 may be biased toward the second position, such that when an applied force is removed from the anchor 500, the tab 610 and the first barb 620 automatically revert to the second position.

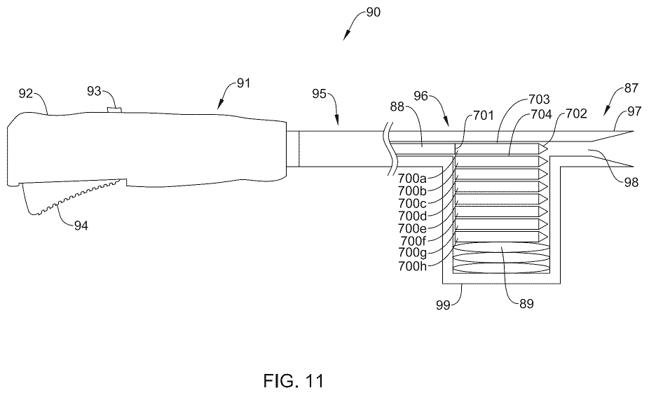

FIG. 11 illustrates an exemplary anchor delivery device 90, such as a tendon stapler. The delivery device 90 may include a handle 91 coupled to a shaft assembly 95. The shaft assembly 95 may include an elongate outer shaft 96 having a longitudinal axis. The handle 91 may include a variety of components which are designed to manipulate an actuation assembly (not shown) located in the outer shaft 96. The outer shaft 96 may include a lumen 98 extending therein to a distal end 87 of the elongate outer shaft 96. The outer shaft 96 may include one or more tines 97 extending away from the distal end 87 of the outer shaft 96. The tines 97 may extend parallel to a central longitudinal axis of the outer shaft 96 in some instances. Although it is not fully shown in FIG. 11, the actuation assembly may be positioned within the lumen 98 of the outer shaft 96. The actuation assembly may include an actuation pusher 88 which extends within the lumen 98 of the outer shaft 96 and into the handle 91.

FIG. 11 further illustrates that the handle 91 may include an actuation mechanism, such as a lever 94 and a thumb trigger 93 for actuating the actuation pusher 88. While the actuation mechanism is illustrated as including a lever and a thumb trigger, it is noted that other forms of actuator may be utilized for the actuation mechanism to manipulate the actuation pusher 88 during use. For instance, in some instances the actuation mechanism may include only one of a lever or thumb trigger. It can be appreciated that the handle 91 may be designed such that a clinician may grasp the handle 91 with one hand and actuate both the lever 94 (via squeezing) and the thumb trigger 93 (via manipulation with the thumb of the grasping hand).

The delivery device 90 may include a tissue anchor magazine 99 coupled to the elongate outer shaft 96. The tissue anchor magazine 99 may house a plurality of tissue anchors 700a, 700b, 700c, 700d, 700e, 700f, 700g, 700h (hereinafter generally referenced as tissue anchors 700). The tissue anchor 700a may be considered to be a leading one of the plurality of tissue anchors 700. The tissue anchors 700 may be an example of tissue anchors 100, 200, 300, 400, 500, 600, described above. Using the tissue anchor 700a as an example, the tissue anchors 700 may each include proximal end 701, a distal end 702, a front side 703, and a back side 704. The tissue anchors 700 may be arranged sequentially within the tissue anchor magazine 99 in a first, pre-deployed configuration (as described above with reference to tissue anchors 100, 200, 300, 400, 500, 600). Thus, the tissue anchors 700 may be arranged within the tissue anchor magazine 99 such that the back side (e.g., back side 704) of the leading one (e.g., 700a) of the plurality of tissue anchors 700 is facing the front side of an adjacent one of the plurality of tissue anchors 700 (e.g., tissue anchor 700b). Adjacent ones of the plurality of tissue anchors 700 are similarly arranged with the back side of one tissue anchor facing the front side of an adjacent tissue anchor 700. The leading one 700a of the plurality of tissue anchors 700 may be nested within the lumen 98 of the elongate outer shaft 96 and be aligned along the longitudinal axis of the outer shaft 96 and/or pusher 88 prior to deployment. The front side of the adjacent one of the plurality of tissue anchors 700 may face the longitudinal axis of the outer shaft 96 and/or the pusher 88, and may move toward the longitudinal axis after deployment of the leading one 700a of the plurality of tissue anchors 700. While it is shown that there are eight tissue anchors 700 within the tissue anchor magazine 99, it may be contemplated that the tissue anchor magazine 99 may include four tissue anchors, six tissue anchors, ten tissue anchors, twelve tissue anchors, or any other suitable number of tissue anchors.

The actuation pusher 88 may be positioned within the elongate outer shaft 96, and actuation of the actuation mechanism (e.g., trigger 93 and/or lever 94) may actuate the pusher 88 to move along the longitudinal axis of the elongate outer shaft 96 to engage the proximal end 701 of the leading one 700a of the plurality of tissue anchors 700 to deploy the leading one 700a of the plurality of tissue anchors 700 from the distal end 87 of the elongate outer shaft 96. The actuation assembly (e.g., actuation mechanism, pusher 88) may be configured to deploy the leading one 700a of the plurality of tissue anchors 700 in a distal direction from the distal end 87 of the elongate outer shaft 96. Upon deployment, the leading one 700a of the plurality of tissue anchors 700 transitions from the first, pre-deployed configuration, to the second, deployed configuration (as described above with reference to tissue anchors 100, 200, 300, 400, 500, 600). Further, upon deployment of the leading one 700a of the plurality of tissue anchors 700, the plurality of tissue anchors 700 remaining in the tissue anchor magazine 99 may shift toward the longitudinal axis of such that another one of the plurality of tissue anchors 700 moves within the lumen 98 of the elongate outer shaft 96 for subsequent deployment from the elongate outer shaft 96.

FIG. 11 further illustrates that the delivery device 90 may include a biasing member, such as a spring 89 disposed within or coupled to the tissue anchor magazine 99. The spring 89 or other biasing member may be designed to exert a force on the back side of a last tissue anchor (e.g., tissue anchor 700h) of the plurality of tissue anchors 700 which shifts the tissue anchors 700 toward the longitudinal axis of the outer shaft 96 upon deployment of the leading one of the plurality of tissue anchors 700. Thus, when the leading one 700a of the plurality of tissue anchors 700 has been deployed, the adjacent tissue anchor (e.g., tissue anchor 700b) moves within the lumen 98 of the elongate outer shaft 96 and may be ready for deployment, wherein upon retraction of the pusher 88 proximally, the pusher 88 may engage a proximal end of the tissue anchor to deploy the next tissue anchor (e.g., tissue anchor 700b). It can be appreciated that the sequence of steps described with respect FIG. 11 may be repeated to sequentially deploy and reload multiple tissue anchors without having to reload and/or remove the delivery device 90 from the patient.

As discussed above, a clinician may actuate an actuation mechanism, such as the trigger 93 or the lever 94, which may initiate the actuation of the spring 89 thereby shifting each of the plurality of tissue anchors 700 from the tissue anchor magazine 99 into the lumen 98 of the outer shaft 96, in succession. Upon release, the spring 89 may expand, thereby laterally shifting the tissue anchors 700, and the lead one 700a of the plurality of tissue anchors 700 into the outer shaft 96. The actuation of the actuation mechanism may further manipulate the pusher 88 distally to engage with the proximal end 701 of the tissue anchor (e.g., tissue anchor 700a) to shift the leading one 700a of the plurality of tissue anchors 700 distally, out of the distal end 87 of the outer shaft 96 to deploy the leading one 700a of the plurality of tissue anchors 700.

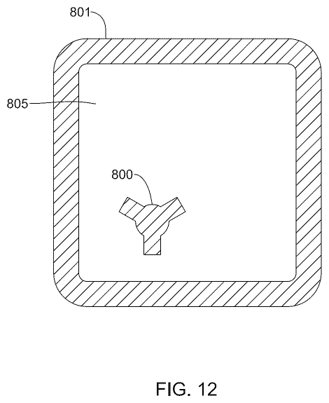

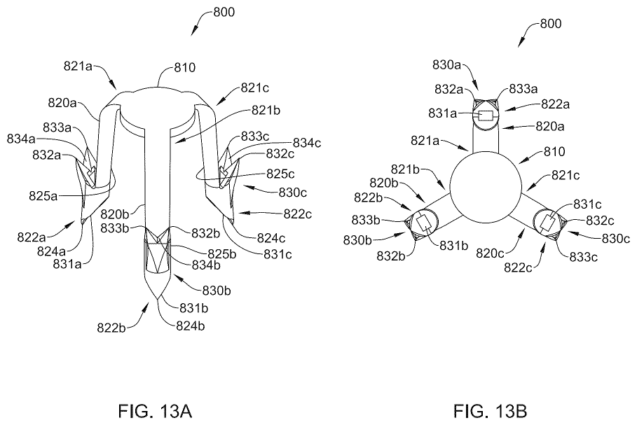

FIG. 12 illustrates a tendon 801 with an implant 805 fixed thereto using an exemplary tissue anchor 800. The tissue anchor 800 will be further described in FIGS. 13A-13B. The implant 805 may include various sheet-like structures. For example, the sheet-like structure may include a plurality of fibers in which the plurality of fibers may be interlinked with one another. In such cases, the sheet-like structure may include a plurality of apertures comprising the interstitial spaces between fibers. As shown in FIG. 12, the tissue anchor 800 may be used to affix the implant 805 to the tendon 801.

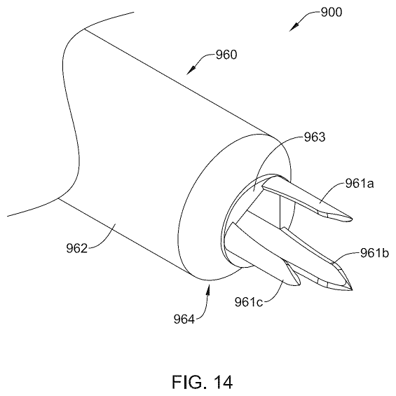

FIGS. 13A-13B illustrate a perspective view of the tissue anchor 800 and a bottom view of the tissue anchor 800, respectively. The tissue anchor 800 may include an anchor body 810. The anchor body 810 may include a flat surface. The flat surface of the anchor body 810 may be a planar surface arranged perpendicular to a longitudinal axis of an outer shaft of a deployment device (as discussed in FIG. 14) when loaded therein. It can be appreciated that the flat surface of the anchor body 810 may define a portion of the tissue anchor 800 for which a pusher (similar to pusher 88) may engage with the tissue anchor 800 to deploy the tissue anchor 800. The tissue anchor 800 may further include a first arm 820a, a second arm 820b, and a third arm 820c. The arms 820a, 820b, 820c may be symmetrically arranged about the anchor body 810. For example, the arms 820a, 820b, 820c may be positioned 120 degrees from each adjacent arm. In some cases, the arms 820a, 820b, 820c may be asymmetrically arranged around the anchor body 810.

The first arm 820a may include a proximal end 821a and a distal end 822a, the second arm 820b may include a proximal end 821b and a distal end 822b, and the third arm 820c may include a proximal end 821c and a distal end 822c. The proximal ends 821a, 821b, 821c may be coupled to the anchor body 810, and the first arm 820a, the second arm 820b, and the third arm 820c may extend distally from the anchor body 810 to the distal ends 822a, 822b, 822c, respectively. The proximal ends 821a, 821b, 821c may be coupled to the anchor body 810 via heat, laser, or sonic welding, injection molding, adhesive, or any other suitable method of attachment. In other instances, the arms 820a, 820b, 820c may be integrally formed with the anchor body 810 and extend therefrom such that the anchor 800 is a monolithic structure including the anchor body 810 and the arms 820a, 820b, 820c attached or joined thereto.

In some cases, the distal end 822a of the first arm 820a may also include a first prong 830a, the distal end 822b of the second arm 820b may include a second prong 830b, and the distal end 822c of the third arm 820c may include a third prong 830c. The prongs 830a, 830b, 830c may each include a distal end 824a, 824b, 824c and a proximal end 825a, 825b, 825c, respectively. In some cases, the distal ends 824a, 824b, 824c may be pointed (e.g., a sharpened, beveled tip) to help pierce tissue upon deployment of the tissue anchor 800. In some cases, the prongs 830a, 830b, 830c may each include a first projection 832a, 832b, 832c and a second projection 833a, 833b, 833c, on each of the first prong 830a, the second prong 830b, and the third prong 830c, respectively. The first projection 832a, 832b, 832c and the second projection 833a, 833b, 833c, on each of the first prong 830a, the second prong 830b, and the third prong 830c, respectively, may extend out and away from the first arm 820a, the second arm 820b, and the third arm 820c, respectively. Having the first projection 832a, 832b, 832c and the second projection 833a, 833b, 833c, on each of the first prong 830a, the second prong 830b, and the third prong 830c, respectively, extend out and away may permit the tissue anchor 800 to engage with tissue, such as tendon tissue, after the tissue anchor 800 is deployed through an implant and into the tissue, such as tendon tissue.

FIGS. 13A-13B further illustrate that the first projection 832a and the second projection 833a of the first prong 830a may define a first notch 834a positioned between the first projection 832a and the second projection 833a, the first projection 832b and the second projection 833b of the second prong 830b may define a second notch 834b positioned between the first projection 832b and the second projection 833b, and the first projection 832c and the second projection 833c of the third prong 830c may define a third notch 834c positioned between the first projection 832c and the second projection 833c.

The prongs 830a, 830b, 830c may each include an aperture extending therein, as depicted by dashed lines extending from the distal ends 824a, 824b, 824c to the proximal ends 825a, 825b, 825c of each of the prongs 830a, 830b, 830c, respectively. For example, a first aperture 831a is located in the first prong 830a, a second aperture 831b is located in the second prong 830b, and a third aperture 831c is located in the third prong 830c. In some cases, the apertures 831a, 831b, 831c may open out on a distal surface at the distal ends 824a, 824b, 824c, respectively, and the apertures 831a, 831b, 831c may open out on a proximal surface at the proximal ends 825a, 825b, 825c of the prongs 830a, 830b, 830c, such that the apertures 831a, 831b, 831c extend entirely through the prongs 830a, 830b, 830c. In some cases, the apertures 831a, 831b, 831c may not extend entirely through the prongs 830a, 830b, 830c, but rather may be a blind hole extending into but not through the prongs 830a, 830b, 830c. The apertures 831a, 831b, 831c may each be configured to receive a tine of a tissue anchor delivery device therein.

Although the various parts of exemplary tissue anchor 800 are depicted in relative proportion to other parts of tissue anchor 800, other configurations in size and orientation of the various parts are also contemplated in other examples.

FIG. 14 illustrates a portion of an exemplary anchor delivery device 900. In particular, FIG. 14 illustrates a shaft assembly 960 of the anchor delivery device 900. The shaft assembly 960 may include a portion of the anchor delivery device 900 which extends distally away from a handle. Additionally, FIG. 14 illustrates that the shaft assembly 960 may include an elongate outer shaft 962, which may be an elongate tubular member. The outer shaft 962 may include an outer surface and a lumen extending therein. Additionally, a distal end region 964 of the outer shaft 962 may include a distal face 963. The distal face 963 may include a surface of the outer shaft 962 which is substantially perpendicular to the longitudinal axis of the outer shaft 962.

Additionally, FIG. 14 illustrates that the shaft assembly 960 may include a plurality of tines 961a, 961b, 961c extending distally from the distal face 963. Collectively, the plurality of tines 961a, 961b, 961c may define a passage through which a tissue anchor (e.g., tissue anchor 800) may pass through as the tissue anchor is deployed out of the outer shaft 962 between the tines 961a, 961b, 961c. Further, the plurality of tines 961a, 961b, 961c may be designed such that they create a pilot hole within the target site tissue. For example, after a clinician aligns the distal end region 964 of the shaft assembly 960 along an implant (e.g., implant 805), the clinician may apply a force to the outer shaft 962 such that the plurality of tines 961a, 961b, 961c pierce through the implant and into the target site (e.g., tendon tissue), thereby creating a pilot hole for which a tissue anchor (e.g., staple) may be inserted.

FIG. 14 illustrates that the plurality of tines 961a, 961b, 961c may include curved sides (e.g., concave surfaces facing the opposing tines 961a, 961b, 961c of the plurality of tines 961a, 961b, 961c) and a pointed end. In some examples, the curved sides of the plurality of tines 961a, 961b, 961c may be configured to mate with curved sides of a variety of example tissue anchors. In different examples, the plurality of tines 961a, 961b, 961c may take various shapes, such as spikes, spears, prongs, or other shapes. Whatever shape the plurality of tines 961a, 961b, 961c may take, they may generally have pointed distal ends for piercing tissue or bone.

The tissue anchor 800 may be loaded within the outer shaft 962 of the shaft assembly 960 such that the arms 820a, 820b, 820c of the tissue anchor 800 are aligned with the tines 961a, 961b, 961c, respectively. The shaft assembly 960 of the anchor delivery device 900 may also include a pusher (not shown) to deploy the tissue anchor 800 from the anchor delivery device 900. The pusher may include first, second, and third tines circumferentially aligned with the arms 820a, 820b, 820c such that the first, second, and third tines of the pusher extending into or through the apertures 831a, 831b, 831c, respectively. Engagement of the tines of the pusher with the apertures 831a, 831b, 831c may assist in orienting the tissue anchor 800 and/or advancing the tissue anchor 800 into tissue.

Tissue anchors 100, 200, 300, 400, 500, 600, 800, may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly (alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly (styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In some embodiments, the tissue anchors 100, 200, 300, 400, 500, 600, 800, or portions thereof, may be made from bioabsorbable or bioerodible materials. Examples of suitable bioabsorbable or bioerodible materials may include bioabsorbable polymers (e.g., polylactic acid (PLA), poly-L-lactic acid (PLLA), poly (lactide-co-glycolide) (PLGA), poly-L-lactide-DL-lactic acid (PLDL)) or a bioerodible metal (e.g., magnesium alloy).

In some embodiments, a degree of Magnetic Resonance Imaging (MRI) compatibility is imparted into the tissue anchors. For example, tissue anchors 100, 200, 300, 400, 500, 600, 800, or portions thereof, may be made of a material that does not substantially distort the image and create substantial artifacts (i.e., gaps in the image). Certain ferromagnetic materials, for example, may not be suitable because they may create artifacts in an MRI image. Tissue anchors 100, 200, 300, 400, 500, 600, 800 or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035such as MP35-N® and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A tissue anchor comprising:
an anchor body having a proximal end and a distal end, wherein the distal end includes a distal tip;
a tab positioned near the proximal end of the anchor body, wherein the tab is movable from a first position to a second position; and
a first barb positioned near the distal end of the anchor body, wherein the first barb is movable from a first position to a second position;
wherein the first barb is biased toward the second position, such that when an applied force is removed from the first barb, the first barb automatically reverts to the second position.

2. The tissue anchor of claim 1, wherein the first barb is in alignment with the anchor body when in the first position.

3. The tissue anchor of claim 1, wherein the first barb deflects away from the anchor body when in the second position.

4. The tissue anchor of claim 1, wherein the tab is in alignment with the anchor body when in the first position.

5. The tissue anchor of claim 1, wherein the tab deflects away from the anchor body when in the second position.

6. The tissue anchor of claim 5, wherein the tab is biased toward the second position, such that when an applied force is removed from the tab, the tab automatically reverts to the second position.

7. The tissue anchor of claim 1, wherein when the tab is in the second position and the first barb is in the second position, the tab deflects away from the anchor body in a first direction and the first barb deflects away from the anchor body in a second direction different from the first direction.

8. The tissue anchor of claim 1, wherein a second barb is positioned near the distal end of the anchor body, wherein the second barb includes a first position in which the second barb is in alignment with the anchor body, and a second position in which the second barb deflects away from the anchor body.

9. The tissue anchor of claim 8, wherein the second barb is biased toward the second position, such that when an applied force is removed from the second barb, the second barb automatically reverts to the second position.

10. The tissue anchor of claim 8, wherein the first and second barbs are located on opposite sides of the anchor body.

11. The tissue anchor of claim 1, wherein the tissue anchor is a monolithic structure and the tab includes a first base end and a second free end, the first base end being joined to the anchor body, and the first barb includes a first base end and a second free end, the first base end being joined to the anchor body.

12. The tissue anchor of claim 1, wherein the first barb is cut out from the anchor body such that the anchor body includes an opening, wherein the first barb is located within a perimeter of the opening in the first position and the first barb extends outward from an opening in the second position.

13. A tissue anchor delivery system, the tissue anchor delivery system comprising:
an elongate shaft having a longitudinal axis, the elongate shaft having a lumen extending therein to a distal end of the elongate shaft;
a tissue anchor magazine coupled to the elongate shaft;
a plurality of tissue anchors each including a front side and a back side, the plurality of tissue anchors arranged sequentially within the tissue anchor magazine in a first, pre-deployed configuration; and
an actuation assembly configured to deploy a leading one of the plurality of tissue anchors in a distal direction from the distal end of the elongate shaft;
wherein upon deployment, the leading one of the plurality of tissue anchors transitions from the first, pre-deployed configuration to a second, deployed configuration.

14. The delivery system of claim 13, wherein the plurality of tissue anchors are arranged front-to-back within the tissue anchor magazine such that the back side of the leading one of the plurality of tissue anchors is facing the front side of an adjacent one of the plurality of tissue anchors.

15. The delivery system of claim 14, wherein the front side of the adjacent one of the plurality of tissue anchors faces the longitudinal axis and moves toward the longitudinal axis after deployment of the leading one of the plurality of tissue anchors.

16. The delivery system of claim 13, wherein the actuation assembly includes a pusher positioned within the lumen of the elongate shaft and a trigger, wherein actuation of the trigger actuates the pusher to move along a longitudinal axis to engage the proximal end of the leading one of the plurality of tissue anchors to deploy the leading one of the plurality of tissue anchors.

17. The delivery system of claim 16, wherein upon deployment of the leading one of the plurality of tissue anchors, the plurality of tissue anchors remaining in the tissue anchor magazine shift toward the longitudinal axis of the pusher such that another one of the plurality of tissue anchors moves within the lumen of the elongate shaft.

18. The delivery system of claim 13, wherein each of the plurality of tissue anchors includes:
an anchor body having a proximal end and a distal end, wherein the distal end includes a distal tip;
a tab positioned near the proximal end of the anchor body; and
a first barb positioned near the distal end of the anchor body, wherein the tab and the first barb move relative to the anchor body when transitioning from the first, pre-deployed configuration to the second, deployed configuration.

19. A tissue anchor comprising:
an anchor body;
a first arm having a proximal end and a distal end, a second arm having a proximal end and a distal end, and a third arm having a proximal end and a distal end, wherein the proximal ends of the first, second, and third arms are coupled to the anchor body and the first, second, and third arms extend distally from the anchor body;
a first prong connected to the distal end of the first arm, the first prong having an aperture extending therein;
a second prong connected to the distal end of the second arm, the second prong having an aperture extending therein; and
a third prong connected to the distal end of the third arm, the third prong having an aperture extending therein;
wherein the first arm, the second arm, and the third arm are symmetrically arranged about the anchor body.

20. The tissue anchor of claim 19, wherein a distal end of the first prong is pointed, a distal end of the second prong is pointed, and a distal end of the third prong is pointed.

21. The tissue anchor of claim 19, wherein the aperture of the first prong, the aperture of the second prong, and the aperture of the third prong are each configured to receive a tine of a tissue anchor delivery device therein.
